(19) United States
(12) Patent Application Publication
(10) Pub. No.: US 2014/0057365 A1
(43) Pub. Date: Feb. 27, 2014

(54) TEST DEVICE AND METHOD FOR COLORED PARTICLE IMMUNOASSAY

(71) Applicant: Jung Moon Ko, Geyonggi-do (KR)

(72) Inventor: Jung Moon Ko, Geyonggi-do (KR)

(73) Assignee: NAVIBIOTECH CO. LTD., Chungcheongnam-do (KR)

(21) Appl. No.: 13/978,208

(22) PCT Filed: Mar. 21, 2012
(86) PCT No.: PCT/KR2012/002035

§ 371 Date: Oct. 28, 2013

(30) Foreign Application Priority Data
Mar. 15, 2012 (KR) 10-2012-0026746

(51) Int. Cl. G01N 33/558

(52) U.S. Cl. CPC G01N 33/558
USPC 436/501; 422/69; 29/527.1

(57) ABSTRACT

A test device and method for colored particle immunoassay. A colorimetric monoclonal antibody is disposed on a receiving piece having a simple structure or in an entrance of a through-hole of a transparent tubular body, such that the monoclonal antibody bonded to micro gold very rapidly reacts with an antigen of a liquid sample. The liquid sample can arrive at a C site and a T site without either a filter or a permeable material having capillary tubes. The C site and the T site are disposed on the interior of the through-hole. This simplifies the structure, thereby reducing the cost of manufacture. The liquid sample can rapidly arrive at the C site and the T site through the through-hole of the transparent tubular body without, so that antibodies of the C site and the T site can react with the antigen in a very short time.

### TECHNICAL FIELD

The present invention relates, in general, to a test device and method for colored particle immunoassay and, more particularly, to a test device and method for colored particle immunoassay in which a liquid sample can directly arrive at a control site or a test site in order to diagnose pregnancy, blood transmitted infectious diseases, sexually transmitted infectious diseases, malaria, dengue fever, tuberculosis, cardiovascular diseases, cancers or the like.

### BACKGROUND ART

As is well known in the art, a test device and method for colored particle immunoassay was proposed in U.S. Pat. No. 6,485,982 (titled “TEST DEVICE AND METHOD FOR COLORED PARTICLE IMMUNOASSAY,” hereinafter referred to as ‘cited invention’). This patent describes a test cell and method for detecting a ligand selected from a liquid sample such as a body fluid.

The test cell includes an elongate outer casing, which houses therein an interior permeable material capable of transporting a test volume and a reservoir volume to an aqueous solution that is applied to an inlet.

The reservoir volume is disposed in a part of the test cell that is away from the inlet, and is filled with an absorbent material. A reservoir acts to receive liquid transported along a flow path defined by the permeable material, and expands along the flow path that extends from the inlet.

In addition, in the test volume, there is a test site which includes a first protein having a binding site specific to a first epitope of the ligand immobilized in fluid communication with the flow path. The test site can be observed through a window of the casing.

An example of the test device in the cited invention is shown in FIG. 1 and FIG. 2. As shown in these figures, in the cited invention, when the liquid sample is absorbed to a downstream flow section 20 which serves as a filter, monoclonal antibodies immobilized at gold particles and antigens of the liquid sample react, so that a color is produced. Consequently, the liquid sample is transported toward a linear antibody formed at the control site by capillary action. The linear antibody formed at the control site and the antigen of the liquid sample then react, so that a color is produced, thereby indicating a normal operation. In consequence, the antibody at the test site reacts or competes with the antigen of the liquid sample, so that a color is produced or no reaction occurs, thereby indicating positive or negative.

In the cited invention, different multiple monoclonal antibodies are required to be attached to the C and T sites, and a filter and a permeable material which contains red-purple micro gold particles therein are required to be used in order to move the liquid sample to the C and T sites by capillary action.

Since a large portion is absorbed to the permeable material before the liquid sample arrives at the C and T sites, the minimum amount of the liquid sample required is increased, which is problematic. Another problem is that the burden of the cost for manufacture of the permeable material increases.

Furthermore, in the cited invention, the input liquid sample must arrive at the C and T sites after having passed through the filter and the permeable material by capillary action. Thus, it is required to wait for at least three minutes in order to obtain a reliable reaction result. Consequently, it is difficult to obtain a rapid result, which is problematic.

### DISCLOSURE

### Technical Problem

Accordingly, the present invention has been made keeping in mind the above problems occurring in the related art, and is intended to provide a test device and method for colored particle immunoassay, in which a liquid sample input into the test device for colored particle immunoassay can arrive at a C site and a T site without either a filter or a permeable material having capillary tubes.

The present invention is also intended to provide a test device and method for colored particle immunoassay, in which a colorimetric monoclonal antibody is disposed on a receiving piece which is additionally disposed with a simple structure or in an entrance of a through-hole of a transparent tubular body, such that the monoclonal antibody bonded to micro gold very rapidly reacts with an antigen of a liquid sample, thereby reducing the cost of manufacture, and the liquid sample can rapidly arrive at the C site and the T site through the through-hole of the transparent tubular body without spending time unlike through the filter and the permeable material, so that antibodies of the C site and the T site can react with the antigen in a very short time.

Furthermore, present invention is also intended to provide a test device and method for colored particle immunoassay, in which the liquid sample can directly arrive at the C site and the T site without being absorbed to and lost by the filter or the permeable material, so that a relatively large amount of the liquid sample can arrive at the C and T sites. This can consequently guarantee a test result using a small amount of the liquid sample.

### Technical Solution

In an aspect, the present invention provides a test device for colored particle immunoassay, which includes a C site and a T site to which two types of multiple monoclonal antibodies are attached and a colorimetric monoclonal antibody to which micro gold is bonded, such that an input liquid sample produces a color at the colorimetric monoclonal antibody and sequentially arrives at the C site and the T site.

A transparent tubular body having a through-hole is prepared, and the C site and the T site are attached to the inside wall of the through-hole. A receiving piece is connected to the front end of the through-hole of the transparent tubular body, and a pressure cap is positioned in the front portion of the transparent tubular body and is coupled to the receiving piece.

### Advantageous Effects

According to the present invention as described above, only when the pressure cap is wrapped on the transparent tubular body, the liquid sample can rapidly react with the colorimetric monoclonal antibody and arrive at the T site and the C site, thereby obtaining a reaction result. Consequently, a test result can be rapidly obtained in several seconds, which is advantageous.

In addition, the present invention can reduce the cost of manufacture without having to use the filter or the permeable material. Substantially no liquid sample is lost by being absorbed to the filter or the permeable material. Therefore, a test result having high reliability can be obtained using a minimum amount of the liquid sample, which is extremely useful.

### DESCRIPTION OF DRAWINGS

FIG. 1 and FIG. 2 are cross-sectional views showing a test device for colored particle immunoassay using a filter and a permeable material of the related art;

FIG. 3 is a perspective view showing a test device for colored particle immunoassay according to the present invention;

FIG. 4 is a longitudinal view showing the structure of the test device for colored particle immunoassay according to the present invention;

FIG. 5 is a perspective view showing an embodiment of the test device for colored particle immunoassay according to the present invention, which prevents a liquid sample from being lost;

FIG. 6 is a longitudinal cross-sectional view showing an embodiment of the test device for colored particle immunoassay according to the present invention, in which a colorimetric monoclonal antibody is positioned at a front end of a through-hole;

FIG. 7 is a longitudinal cross-sectional view showing an embodiment of the test device for colored particle immunoassay according to the present invention, which prevents a liquid sample from flowing out;

FIG. 8 is a longitudinal cross-sectional view showing an embodiment of the test device for colored particle immunoassay according to the present invention, which prevents a liquid sample from flowing out and discharges an increased pressure using a pressure cap;

FIG. 9 is a longitudinal cross-sectional view showing an embodiment of the test device for colored particle immunoassay according to the present invention, which employs an O-ring for increasing a positive pressure;

FIG. 10 is a longitudinal cross-sectional view showing an embodiment of the test device for colored particle immunoassay according to the present invention, which decreases an excessive positive pressure;

FIG. 11 and FIG. 12 are longitudinal cross-sectional views showing an embodiment of the test device for colored particle immunoassay according to the present invention, which employs a handle;

FIG. 13 is a perspective view showing an embodiment of the test device for colored particle immunoassay according to the present invention, which has an oblong through-hole; and

FIG. 14 is a longitudinal view showing the test device for colored particle immunoassay according to the present invention, in which a T site and an S site are separately disposed in upper and lower portions of a through-hole.

FIG. 15 is a flowchart showing a test method for colored particle immunoassay according to the present invention.

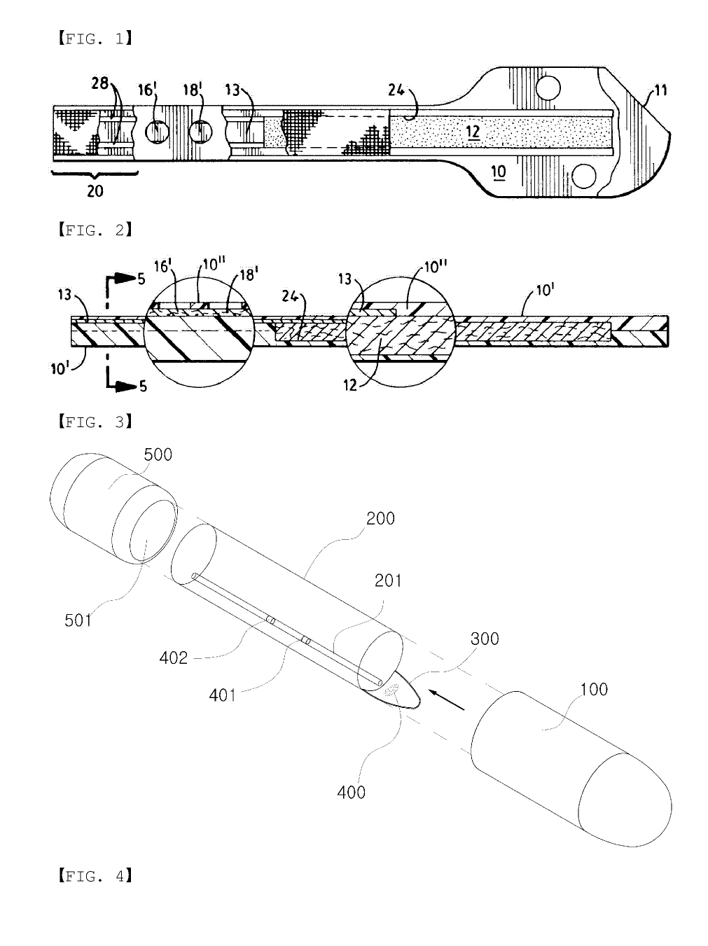

### MAJOR REFERENCE NUMERALS AND SYMBOLS OF THE DRAWINGS

| | |
|---|---|
| 100: pressure cap | 101: recess |
| 102: pressure-reducing hole | 103: O-ring |
| 200: transparent tubular body | 201: through-hole |
| 202: discharge hole | 300: receiving piece |
| 301: recessed portion | 302: guide wall |
| 400: colorimetric monoclonal antibody | |
| 401: T site | 402: C site |
| 500: handle | 501: recessed groove |
| 600: absorbent material | |

### BEST MODE

The present invention provides a test device for colored particle immunoassay in which an extracted liquid sample is caused to pass through a colorimetric monoclonal antibody which is fixed to micro gold and a C site and a T site to which two types of multiple monoclonal antibodies are attached, so that the liquid sample specifically reacts with an antibody and produces a color at the C site. The test device includes a transparent tubular body having a through-hole, the C site and the T site being attached to an inside wall of the through-hole, a receiving piece connected to a front end of the through-hole in a front portion of the transparent tubular body, and a pressure cap positioned in front of the transparent tubular body and coupled to the receiving piece.

Also provided is a test method for colored particle immunoassay which includes: a molding step of molding a transparent tubular member having a through-hole; a receiving piece-forming step of providing a receiving piece in a portion of the transparent tubular member; a step of arranging a colorimetric monoclonal antibody in which micro gold having a monoclonal antibody fixed thereto is arranged on the receiving piece or in the through-hole in a front end of the transparent tubular member; and an antibody attachment step of attaching two types of multiple monoclonal antibodies having a shape of a belt to a C site and a T site which are allotted to the through-hole inside the transparent tubular body, thereby preparing a test device for colored particle immunoassay.

The test method also includes a reaction step of placing an extracted liquid sample on the receiving piece and, under a positive pressure that is produced by a pressure cap which is fitted into a portion of the receiving piece, allowing the liquid sample that has reacted with the colorimetric monoclonal antibody of the micro gold on the receiving piece to flow into the through-hole inside the transparent tubular body, be condensed and produce a color at the C site, and arrive at the T site, so that whether or not a specific antigen is present in the liquid sample is determined; and after the reaction step, a residue absorption step of allowing the liquid sample that is discharged through the rear end of the through-hole of the transparent tubular body to be absorbed to an absorbent material disposed in the rear end of the through-hole.

### MODE FOR INVENTION

Reference will now be made in detail to various embodiments of the present invention, examples of which are illustrated in the accompanying drawings and described below, so that a person having ordinary skill in the art to which the present invention relates can easily put the present invention into practice.

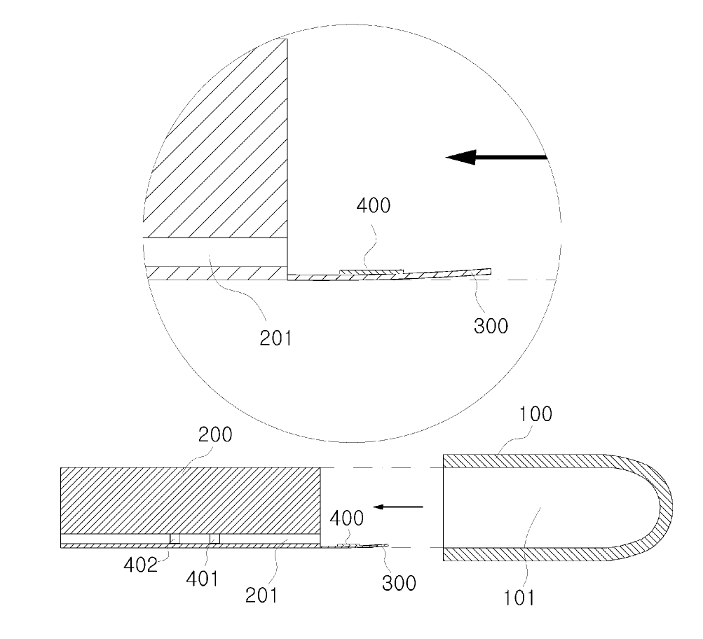

FIG. 3 and FIG. 4 show the basic outer shape and structure of a test device according to the present invention.

As shown in these figures, the present invention includes a pressure cap 100 made of an elastic material and a transparent tubular body 200 which is coupled to a recess 101 of the pressure cap 100, the transparent tubular body 200 having defined a through-hole 201 therein.

In the present invention, in the transparent tubular body 200, a receiving piece 300, the through-hole 201 and a colorimetric monoclonal antibody 400 are disposed. The receiving piece 300 has a recessed portion 301 in a front end thereof such that a small amount of liquid sample can be collected therein. The through-hole 201 communicates with the recessed portion 301 of the receiving piece 300 and extends toward the rear end. The colorimetric monoclonal antibody 400 is made of microscopic gold, in which a monoclonal antibody is bonded to the recessed portion 301 of the receiving piece 300. In addition, a C site 402 and a T site 401, to which two types of multiple monoclonal antibodies are respectively attached, are provided inside the through-hole 201.

A following detailed description will be given of an example of the present invention applied to a pregnancy test kit.

According to the present invention, first, for a pregnancy test, hCG in blood or urine discharged form a pregnant woman is intended to react as an antigen. For this, one type of monoclonal antibody that is specifically bonded to hCG is produced from a rat. Afterwards, the colorimetric monoclonal antibody 400 is disposed by attaching it to red-purple microscopic gold particles fixed to the recessed portion 301 of the receiving piece 300. Multiple monoclonal antibodies from a rat or goat that can be bonded to hCG are immobilized in the shape of a linear belt at the T site 401 of the transparent tubular body 200.

In addition, antibodies which produce color by reacting with all types of antigens are immobilized in the shape of a linear belt at the C site 402 of the transparent tubular body 200.

After that, when blood or urine from a pregnant woman is applied to the receiving piece 300, the colorimetric monoclonal antibody 400 bonded to the micro gold of the receiving piece 300 is bonded to hCG, thereby producing a red color. In this state, when the pressure cap 100 is inserted into transparent tubular body 200, air inside the recessed portion 301 of the pressure cap 100 flows out via the through-hole 201 of the transparent tubular body 200. At the same time, the colorimetric monoclonal antibody 400 bonded to the micro gold that has reacted in the receiving piece 300 also moves and arrives at the T site 402. At the T site 402, the colorimetric monoclonal antibody 400 is bonded to another multiple monoclonal antibody (poly-goat anti-hCG). At this time, the micro gold particles are attached to multiple monoclonal antibodies which are arranged and immobile in the shape of a belt at the T site 402, thereby producing a red-purple color.

In addition, the other antigens except for hCG antigen continuously move under the pressure of the air that passes through the through-hole 201 and arrive at the C site 401. Here, a color is produced due to bonding of the antibody at the C site 401 (anti-mouse IgG) by another antigen irrespective of presence of hCG. Therefore, a function normally occurs, and a result indicates a readable status. A residue is discharged to the rear end of the through-hole 201.

At this time, if hCG is not contained in the blood or urine of the pregnant woman, there is no reaction with the colorimetric monoclonal antibody 400 that is fixed to the micro gold particles disposed in the receiving piece 300 of the transparent tubular body 200. Consequently, even if the micro gold moves, micro gold then passes through the T site 401 without being bonded to the antibody at the T site 401, so that no color is produced. Also in this case, a color is produced at the C site 402, thereby indicating a normal operation, which represents non-pregnancy.

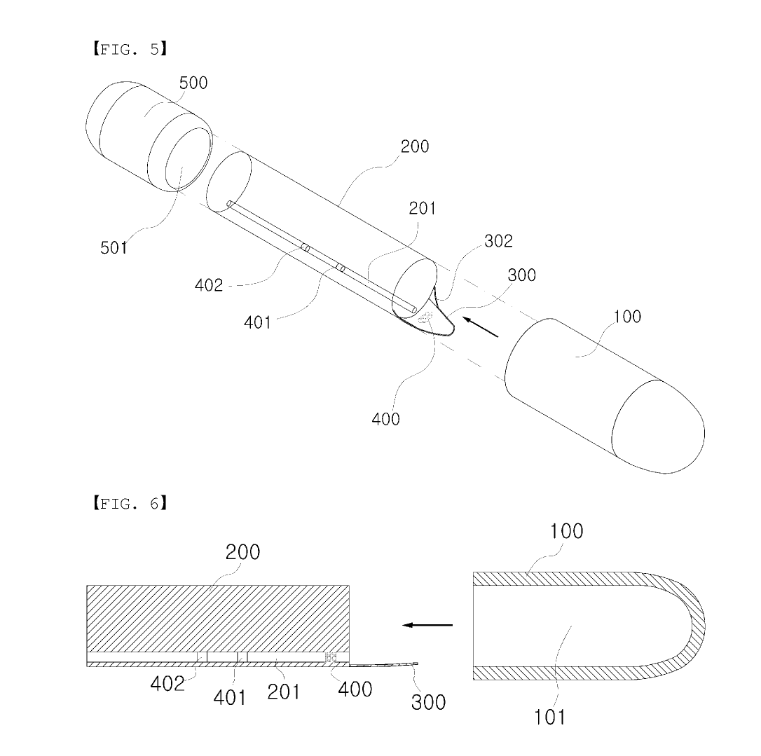

In addition, when a user fails to stably hold the transparent tubular body 200 when using it, the liquid sample may spill without flowing from the receiving piece 300 into the through-hole 201. The present invention is intended to prevent this, and provides guide walls 302 erected on both sides of the receiving piece 300, as shown in FIG. 5.

In addition, as shown in the cross-sectional view of FIG. 6, the colorimetric monoclonal antibody 400 fixed to the micro gold which is specifically bonded to hCG from a rat can be attached to the inside wall of the front end of the through-hole 201 of the transparent tubular body 200 instead of being attached to the receiving piece 300. This configuration can overcome the problem that the arrangement of the micro gold might be unstable due to the small thickness during the process of treatment.

Furthermore, as shown in FIG. 7, according to an embodiment of the present invention, at the moment that the pressure cap 100 is pushed into the transparent tubular body 200, the air inside the pressure cap 100 flows out so that the residue of the liquid sample is discharged through the rear portion of the transparent tubular body 200. In order to prevent the discharged residue from contaminating the working environment or the like, an absorbent material 600 can be disposed inside the through-hole 201.

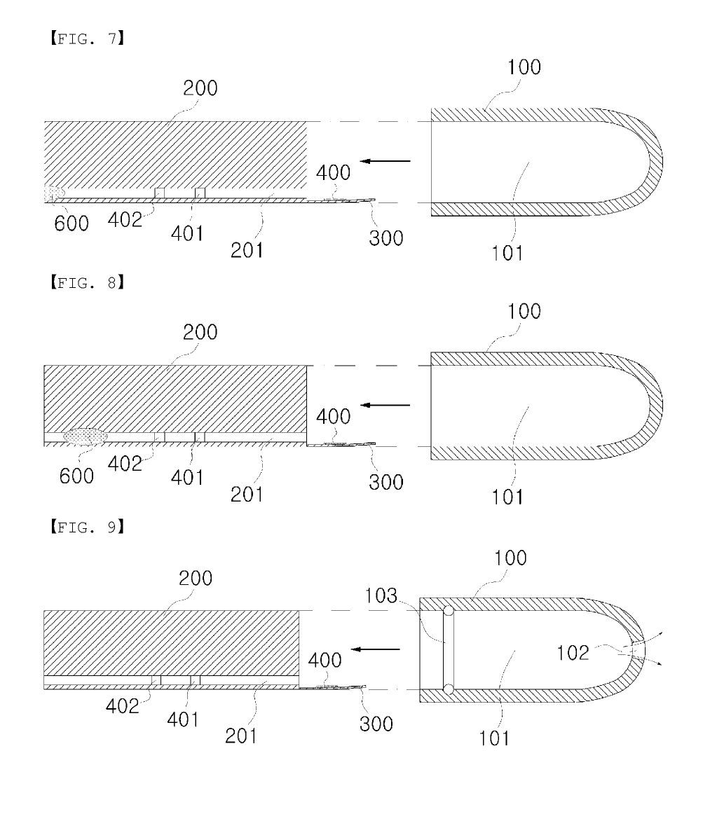

In addition, as shown in FIG. 8, according to an embodiment of the present invention, after the reaction step in which the liquid sample enters the through-hole 201 inside the transparent tubular body 200 in response to the pressure of the pressure cap 100 and sequentially arrives at the C site 402 and the T site 401, the liquid sample can be absorbed to the residue absorbent material 600 and then be discharged through a discharge hole 202.

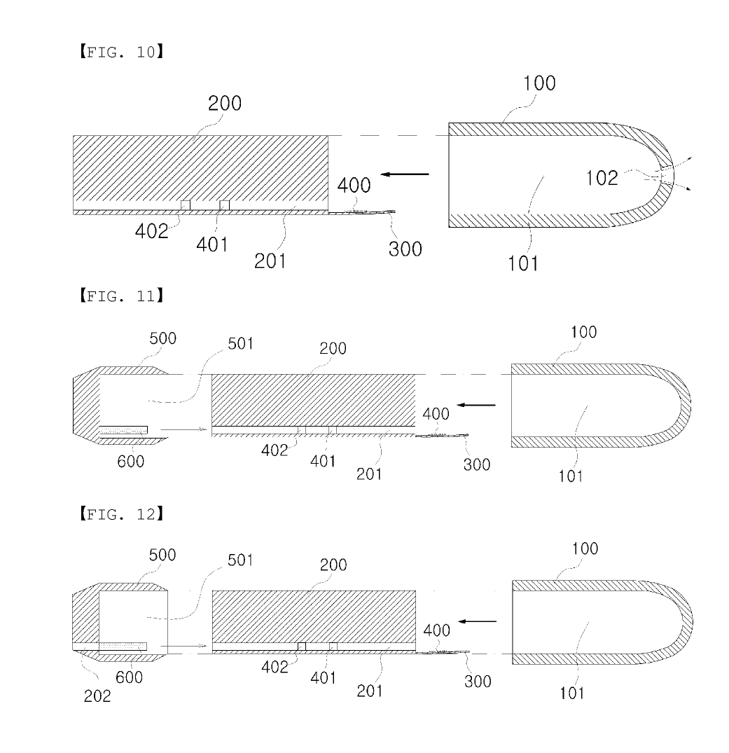

Furthermore, as described above, the present invention uses the pressure of the pressure cap 100 in order to make the liquid sample arrive at the colorimetric monoclonal antibody 400 bonded to the micro gold and at the antibodies immobilized at the T site 401 and the C site 402. As shown in FIG. 9, an O-ring 103 can be disposed on the inner circumference of the pressure cap 100 so that a sufficient amount of pressure is produced in the pressure cap 100.

According to this embodiment, when the pressure cap 100 is closed, all of the air can flow into the through-hole 201 of the transparent tubular body 200 without leakage, thereby increasing the pressure. Consequently, the liquid sample can rapidly arrive at the T site 401 and the C site 402.

In addition, the speed of the liquid sample passing through the through-hole 201 is excessively high when an excessive amount of air is discharged from the pressure cap 100 through the through-hole 201 of the transparent tubular body 200. According to the present invention, as shown in FIG. 9 and FIG. 10, a pressure-reducing hole 102 is formed in one end of the pressure cap 100, such that a portion of the air can leak through the pressure-reducing hole 102 at the moment that the pressure cap 100 is inserted into the transparent tubular body 200. This can reduce the pressing force so that the speed at which the liquid sample flows can be suitably maintained.

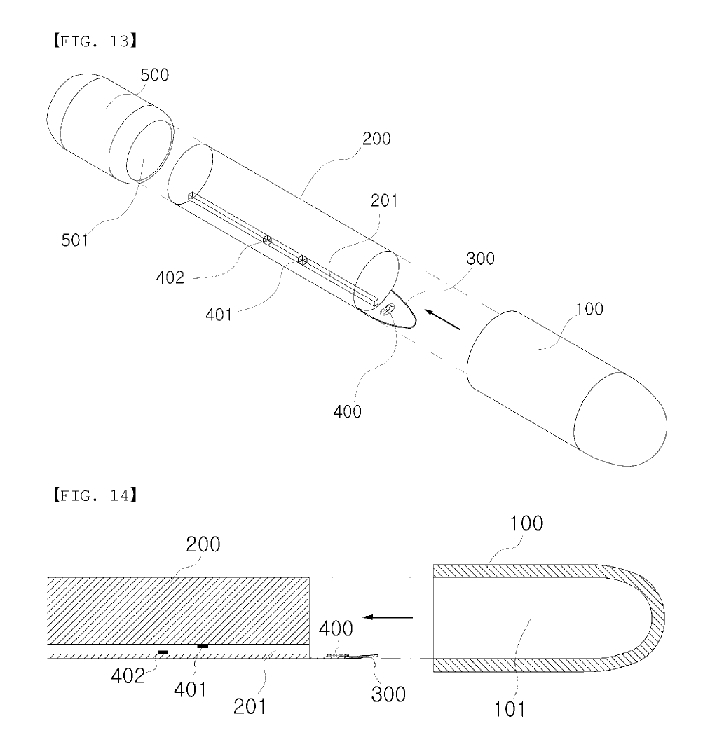

Furthermore, according to the present invention, since no large amount of the liquid sample is discharged through the rear end of the transparent tubular body 200, the absorbent material 600 is internally disposed around a recessed groove 501 of a handle 500 and is fitted into the rear end of the transparent tubular body 200, as shown in FIG. 11. Consequently, the residue that is discharged through the rear end 201 of the transparent tubular body 200 can be absorbed to the absorbent material 600 without being discharged to the outside. This makes sanitary treatment possible.

Also in this case, according to the present invention, since no large amount of the liquid sample is discharged through the rear end of the transparent tubular body 200, the absorbent material 600 is internally disposed around a recessed groove 501 of a handle 500 and is fitted into the rear end of the transparent tubular body 200, as shown in FIG. 12. Consequently, the residue that is discharged through the rear end 201 of the transparent tubular body 200 can be absorbed to the absorbent material 600 before the air pressure is discharged to the discharge hole 202. This can lead to suitable operation and prevent the liquid sample from flowing out, thereby making sanitary treatment possible.

Furthermore, according to the present invention, the cross-section of the through-hole 201 of the transparent tubular body 200 has a circular shape, as shown in FIG. 3. The distances of the multiple monoclonal antibodies of the C site 402 and the T site 401 attached to the inside wall of the through-hole 201 are set in the range from 3 mm to 8 mm so as to be identifiable.

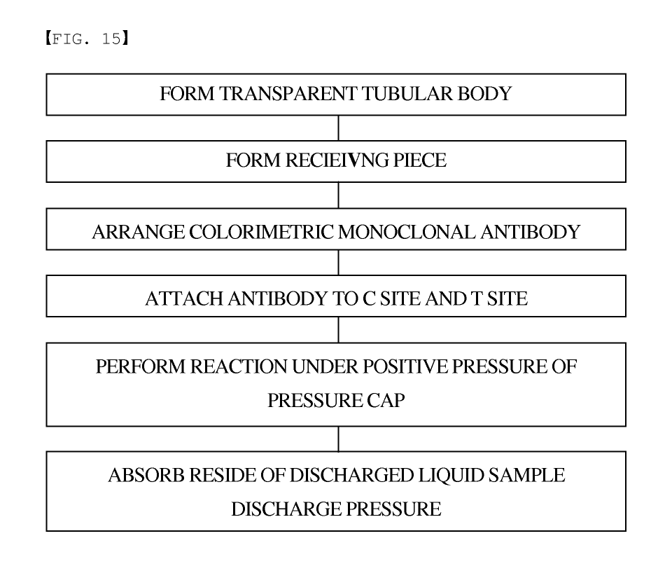

In addition, according to the present invention, the cross-section of the through-hole 201 of the transparent tubular body 200 has an oblong shape, as shown in FIG. 13. As described above, the distances of the multiple monoclonal antibodies of the C site 402 and the T site 401 attached to the inside wall of the through-hole 201 are set in the range from 3 mm to 8 mm so as to be identifiable. In particular, when the cross-section of the through-hole 201 of the transparent tubular body 200 has the circular or oblong shape, the multiple monoclonal antibodies of the C site 402 and the T site 401 are separately attached to the upper and lower portions, as shown in FIG. 14. Consequently, the positions can look significantly different, thereby increasing readability.

Furthermore, as shown in FIG. 15, the present invention is devised such that a liquid sample, or an antigen, is bonded to and reacts with antibodies while moving under a positive pressure that is produced when the pressure cap 100 is coupled to the through-hole 201 of the transparent tubular body 200. The present invention includes a molding step of forming the transparent tubular body 200 having the through-hole 201 by injection or extrusion molding;

a step of forming the receiving section 300 such that the receiving section 300 is attached to the front portion of the transparent tubular body 200 or is molded integrally with the transparent tubular body 200;

a step of arranging the colorimetric monoclonal antibody 400 in which the micro gold having a monoclonal antibody fixed thereto is arranged on the receiving piece 300 at the front portion of the transparent tubular body 200 or in the through-hole 201 in the front end of the transparent tubular body 200; an antibody attachment step of attaching two types of multiple monoclonal antibodies having the shape of a belt to the C site 402 and the T site 401 which are allotted to the through-hole 201 inside the transparent tubular body 200;

a reaction step of placing an extracted liquid sample on the receiving piece 300 and, under a positive pressure that is produced by fitting the pressure cap 100 around the receiving piece 300, allowing the liquid sample that has reacted with the colorimetric monoclonal antibody 400 of the micro gold on the receiving piece 300 to flow into the through-hole 201 inside the transparent tubular body 200, be condensed and produce a color at the C site 402, and arrive at the T site 401, so that whether or not a specific antigen is present in the liquid sample is determined; and after the reaction step, a residue absorption step of allowing the liquid sample that is discharged through the rear end of the through-hole 201 of the transparent tubular body 200 to be absorbed to the absorbent material 600 disposed in the rear end of the through-hole 201.

After the reaction step in which the liquid sample flows into the through-hole 201 inside the transparent tubular body 200 and sequentially arrives at the C site 402 and the T site 401 under the pressure of the pressure cap 100, a discharge step of discharging the pressure through the discharge hole 202 is provided in addition to the residue absorption step of absorbing the residue to the absorbent material 600.

Furthermore, the present invention can additionally include a pressure reducing step of, when the pressure applied to the transparent tubular body 200 by the pressure cap 100 is excessively high, reducing the pressure via the pressure-reducing hole 102 formed in one portion of the pressure cap 100.

In addition, as set forth in the foregoing embodiments of the present invention, the test device and method for colored particle immunoassay which are applied to a pregnancy test kit with which pregnancy is tested based on an antibody which specifically reacts with and is bonded to hCG, an antigen, were described for illustrative purposes. The present invention can also be used for a variety of purposes of diagnosing a variety of diseases, such as blood transmitted infectious diseases, sexually transmitted infectious diseases, malaria, dengue fever, tuberculosis, cardiovascular diseases and cancers. For this, the T site 401 and the S site are fixed to a variety of micro particles including the colorimetric monoclonal antibody 400 which exhibits a reaction specific to a bacterium of a corresponding disease or a variety of cancer markers and, at the same time, are attached in the shape of a belt to the through-hole 201 of the transparent tubular body 200, so that a reaction occurs when the liquid sample is moved using the pressure cap 100.

The present invention that has been set forth above is not limited to the foregoing embodiments, and it is possible to variously carry out the present invention by changing several well-known components and methods without departing from the gist and concept of the present invention.

1. A test device for colored particle immunoassay in which an extracted liquid sample is caused to pass through a colorimetric monoclonal antibody which is fixed to micro gold and a C site and a T site to which two types of multiple monoclonal antibodies are attached, so that the liquid sample specifically reacts with an antibody and produces a color at the C site,
the test device comprising a transparent tubular body having a through-hole, the C site and the T site being attached to an inside wall of the through-hole, a receiving piece connected to a front end of the through-hole in a front portion of the transparent tubular body, and a pressure cap positioned in front of the transparent tubular body and coupled to the receiving piece.

2. The test device according to claim 1, wherein the colorimetric monoclonal antibody bonded to the micro gold is attached to an upper surface of the receiving piece.

3. The test device according to claim 1, wherein the colorimetric monoclonal antibody bonded to the micro gold is attached to the inside wall of the front end of the through-hole of the transparent tubular body.

4. The test device according to claim 1, wherein the receiving piece has a recessed portion.

5. The test device according to claim 1, wherein the receiving piece has guide walls on both sides thereof.

6. The test device according to claim 1, wherein a residue absorbent material is disposed in a rear end of the through-hole of the transparent tubular body.

7. The test device according to claim 6, wherein a discharge hole is formed behind the residue absorbent material which is disposed in the rear end of the transparent tubular body.

8. The test device according to claim 1, wherein an O-ring is disposed on an inner circumference of the pressure cap.

9. The test device according to claim 1, wherein a pressure-reducing hole with which pressure is to be reduced is formed in a portion of the pressure cap.

10. The test device according to claim 1, wherein a handle which is coupled to a rear end of the transparent tubular body is provided.

11. The test device according to claim 10, wherein the handle which is coupled to the rear end of the transparent tubular body receives therein the residue absorbent material.

12. The test device according to claim 11, wherein the handle which receives therein the residue absorbent material in the rear portion of the transparent tubular body has a discharge hole.

13. The test device according to claim 1, wherein the multiple monoclonal antibodies of the C site and the T site attached to an interior of the transparent tubular body are attached to upper and lower portions inside the through-hole and spaced apart from each other by a predetermined distance.

14. (canceled)

15. The test method according to claim 18, further comprising:
a pressure reducing step of, when a pressure applied to the transparent tubular body by the pressure cap is excessively high, reducing the pressure via a pressure-reducing hole formed in one portion of the pressure cap.

16. The test method according to claim 15, further comprising: a discharge step of discharging the pressure through the discharge hole,
said discharge step being performed after the reaction step;
said discharge step being performed in addition to the residue absorption.

17. A method of preparing a test device for colored particle immunoassay, comprising:
a molding step of molding a transparent tubular member having a through-hole;
a receiving piece-forming step of providing a receiving piece in a portion of the transparent tubular member;
a step of arranging a colorimetric monoclonal antibody in which micro gold having a monoclonal antibody fixed thereto is arranged on the receiving piece or in the through-hole in a front end of the transparent tubular member; and
an antibody attachment step of attaching two types of multiple monoclonal antibodies having a shape of a belt to a C site and a T site which are allotted to the through-hole inside the transparent tubular body, thereby preparing said test device.

18. A test method for colored particle immunoassay using the test device prepared by the method of claim 17, comprising:
a reaction step of placing an extracted liquid sample on the receiving piece and, under a positive pressure that is produced by a pressure cap which is fitted into a portion of the receiving piece, allowing the liquid sample that has reacted with the colorimetric monoclonal antibody of the micro gold on the receiving piece to flow into the through-hole inside the transparent tubular body, be condensed and produce a color at the C site, and arrive at the T site, so that whether or not a specific antigen is present in the liquid sample is determined; and
after the reaction step, a residue absorption step of allowing the liquid sample that is discharged through the rear end of the through-hole of the transparent tubular body to be absorbed to an absorbent material disposed in the rear end of the through-hole.

19. A test method for colored particle immunoassay using the test device of claim 1, comprising:
a reaction step of placing an extracted liquid sample on the receiving piece and, under a positive pressure that is produced by the pressure cap, allowing the liquid sample that has reacted with the colorimetric monoclonal antibody of the micro gold to flow into the through-hole inside the transparent tubular body, be condensed and produce a color at the C site, and arrive at the T site, so that whether or not a specific antigen is present in the liquid sample is determined; and
after the reaction step, a residue absorption step of allowing the liquid sample to be discharged through the rear end of the through-hole of the transparent tubular body and absorbed to an absorbent material disposed in the rear end of the through-hole.

20. A test method for colored particle immunoassay using a test device comprising:
a transparent tubular member having a through-hole;
a receiving piece in a portion of the transparent tubular member;
micro gold having a monoclonal antibody fixed thereto arranged on the receiving piece or in the through-hole in a front end of the transparent tubular member; and
two types of multiple monoclonal antibodies having a shape of a belt to a C site and a T site which are positioned in the through-hole inside the transparent tubular body;
wherein said test method comprises:
a reaction step of placing an extracted liquid sample on the receiving piece and, under a positive pressure that is produced by a pressure cap which is fitted into a portion of the receiving piece, allowing the liquid sample that has reacted with the colorimetric monoclonal antibody of the micro gold on the receiving piece to flow into the through-hole inside the transparent tubular body, be condensed and produce a color at the C site, and arrive at the T site, so that whether or not a specific antigen is present in the liquid sample is determined; and
after the reaction step, a residue absorption step of allowing the liquid sample that is discharged through the rear end of the through-hole of the transparent tubular body to be absorbed to an absorbent material disposed in the rear end of the through-hole.
